(19) United States
(12) Patent Application Publication
(10) Pub. No.: US 2026/0243914 A1
(43) Pub. Date: Aug. 20, 2026

(54) SYSTEM AND METHOD FOR HYBRID AIRPORT SECURITY SCREENING WITH VOLUNTARY WALK-THROUGH MEDICAL IMAGING

(71) Applicant: Morteza Naghavi, San Diego, CA (US)

(72) Inventor: Morteza Naghavi, San Diego, CA (US)

(21) Appl. No.: 19/417,345

(22) Filed: Dec. 12, 2025

Related U.S. Application Data
(63) Continuation-in-part of application No. 18/771,542, filed on Jul. 12, 2024.

(51) Int. Cl. G01V 3/12; G01N 23/046

(52) U.S. Cl. CPC G01V 3/12; G01N 23/046; G01N 2223/612

(57) ABSTRACT

A system and method are disclosed for hybrid screening in controlled-access environments that enable voluntary preventive medical imaging without interfering with mandatory security screening operations. The system comprises a non-ionizing security screening subsystem configured to perform mandatory security scans, a walk-through low-dose computed tomography (CT) medical imaging subsystem configured for voluntary preventive screening, a biometric identity verification subsystem, and a workflow orchestration and physical routing system. The system further includes automated height and weight measurement synchronized with CT image acquisition and AI-based analysis for cardiopulmonary or cardiometabolic risk assessment. The invention enables high-throughput, low-dose preventive screening in environments such as airports and other controlled-access facilities without degrading security throughput or compromising privacy, regulatory compliance, or operational efficiency.

### CROSS-REFERENCE

This application is Continuation-In-Part of the U.S. patent application Ser. No. 18/771,542 filed Jul. 12, 2024, the entirety of which is incorporated herein by reference.

### FIELD OF INVENTION

The present invention is improvement of previously filed application relating generally to medical imaging systems and automated walk-through imaging workflows. More particularly, the present invention relates to extending automated walk-through medical imaging systems into airport and controlled-access environments, in coordination with existing security screening procedures, to enable voluntary preventive medical imaging without altering mandatory security screening operations.

### BACK GROUND

As previously disclosed in the parent application, medical imaging scans, such as computed tomography (CT) scans, are essential diagnostic tools in modern healthcare. However, the process of visiting a physician to obtain a physician order, contacting imaging facilities for scheduling, travelling to the facility, filling out forms and waiting to undergo a scan is highly time-consuming and inconvenient for most people. Traditional imaging facilities require patients to schedule appointments well in advance, fill out lengthy questionnaires, and spend considerable time waiting before the actual scanning procedure.

While this may be needed for sick care where extra care is needed for ill individuals, it is unnecessary and prohibitive for screening purposes where asymptomatic or apparently healthy need to undergo imaging for preventive care.

The lengthy and inconvenient process of traditional medical imaging poses significant barriers to adoption for asymptomatic individuals undergoing screening tests for early detection of potentially fatal diseases such as heart disease and lung cancer. These individuals, who are not currently sick, have a much lower tolerance for the extended time commitments required by conventional imaging procedures. As a result, the inconvenience factor becomes a major deterrent to participation in these critical preventive health measures.

Furthermore, automation and reducing the need for human resources can reduce the cost therefore making the imaging test more affordable for a larger portion of the population. Currently less than 10% of lung cancer and less than 5% of heart disease are detected via screening which allows for detecting early stage diseases, the rest are diagnosed because of symptoms which are late-stage and often fatal and costly.

Similar statistics are true for detection of osteoporosis and fatty liver diseases which are almost always found via symptoms. We have previously patented new AI-enabled technologies for rapid detection of all these pathologies namely heart disease, lung cancer, osteoporosis and fatty liver through a chest CT scan (Ref patent applications or our publications see reference at the bottom). Such an AI-enabled tool helps mass adoption of CT-based screening. However, easy access to CT scanning facilities remains a major barrier to mass adoption and public benefit from such an AI-enabled screening tool. In this invention we aim to eliminate this barrier to mass adoption and as well as financial barriers by using automation technology to reduce costs.

Drive-through services have been widely adopted in various industries to enhance convenience and efficiency for customers. For example, drive-through restaurants, pharmacies, and banks have become increasingly popular. However, the concept of drive-through services has not been extensively explored in the field of medical imaging. Nonetheless, the drive-through COVID-19 testing and vaccination during the COVID pandemic provided a glimpse into the feasibility of such an approach to medical services.

A prior art document, US Patent Application Publication No. 20230398028, discloses a drive-thru medical facility that includes a vehicle pathway and a clinic building. The vehicle pathway comprises an entrance, at least one diagnosis bay for receiving vehicles, an intermediate section, a first exit, and at least one parking space. The clinic building is positioned to be accessible by passengers of vehicles parked in the parking spaces. While this prior art document describes a drive-through medical facility, it does not specifically address the challenges associated with performing medical imaging scans in a drive-through setting. For example, the drive-through system described in this invention is enabled via mobile apps and cloud connectivity unlike US Patent Application 20230398028, this invention does not have cash registration desk or physical spaces for medical staff interactions with patients to gather information or to obtain informed consent, all of which are done via an interactive mobile app.

While drive-through services have been implemented in various industries, and some prior art documents describe drive-through medical facilities, there remains a need for a convenient and efficient system and method specifically designed for performing medical imaging scans in a drive-through setting. The present invention addresses this need by providing a novel drive-through medical imaging system and method that streamlines the entire process, from scheduling and preparation to the actual scanning procedure, thereby improving the overall patient experience, operational efficiency and convenience of use of imaging facilities.

Similar rationales are applicable to a Walk-Through imaging system particularly when the Walk-Through system is powered by AI-enabled automation and is linked to the user's mobile app that manages the entire data gathering, communications and transactions all of which are currently done inefficiently through an arduous and time-consuming processes in hospitals and imaging facilities.

The main concern in such a futuristic Drive-Through and Walk-Through imaging facilities would be when the imaging device such as a CT scan requires X-ray which is known as being hazardous. However, with the development of new X-ray tubes that made super low-dose CT scan imaging possible, and the fact that currently human operators only choose from exiting menu of protocols created in the CT scanner and do not decide on the X-ray dose levels rather than simply click on a drop-down button and choose form the available protocols for example a protocol for coronary artery calcium scan or for lung cancer screening scan.

This operation can easily be automated by AI for specialized or solo-function dedicated CT imaging in a drive-through or walk-through CT system that is focused on cardiac and lung cancer screening. It would require a lot of programming to make it a general purpose fully AI controlled imaging versus a highly focused system for high throughput chest CT scanning (lung cancer and heart disease screening) as described here as the main embodiment of the invention.

Therefore, automated drive-through and walk-through medical imaging systems—particularly those using computed tomography (CT)—enable rapid, efficient, and scalable preventive health screening by reducing the need for traditional clinical workflows, onsite medical personnel, and extended appointment scheduling.

Low-dose CT imaging has proven value in early detection of cardiopulmonary disease, including lung cancer and cardiovascular disease, particularly when combined with automated or AI-based image analysis. However, access to CT imaging remains limited by logistical barriers, cost, and physical separation between imaging facilities and everyday environments.

Airports and other controlled-access transportation hubs already process large volumes of individuals through standardized screening procedures. These environments present a unique opportunity to offer voluntary preventive medical imaging to individuals who choose to participate, provided that such imaging does not interfere with, replace, or compromise existing security screening operations.

Existing airport security systems are designed exclusively for threat detection and rely primarily on millimeter-wave or other non-ionizing technologies. These security systems are governed by regulatory frameworks and operational constraints distinct from those applicable to medical imaging. Attempts to merge security screening and medical imaging into a single modality raise substantial technical, regulatory, and privacy challenges.

Accordingly, these remains a need for a system that preserves mandatory security screening using existing modalities, enables voluntary, opt-in medical imaging using walk-through CT systems, maintains clear regulatory and data separation between security and medical functions, and integrates passenger identity, consent, and physical workflow in a scalable manner.

### SUMMARY OF THE INVENTION

The previously disclosed invention provides a drive-through and walk-through medical imaging system that enables efficient and convenient imaging scans for users. The system comprises a facility with guided entrance and exit, a mobile application for qualifying users, scheduling appointments and generating physician orders, a scanner area with a medical imaging device, and for drive-through a parking area for users' vehicles.

The system includes embedding visible light cameras in the scanner area and the scanner itself to enable AI-guided management of scanning without human interventions and to generate combined 3D images resulting from visible light of the body's exterior and X-ray imaging of the body's interior enabling co-registration for a more realistic images used for patient education and interactions including augmented reality tools.

User drives to the facility's entrance, which may include signs and a teller that is either human or an automated AI. The user must have the mobile application downloaded, questions answered, physician order generated, and scan appointment scheduled. A QR code is provided that activates a green sign to guide the user to move forward to the scanner area.

The user is guided to drive forward inside the facility where the medical imaging device, such as a full-body weight-bearing CT enabling a standing scan position, is located inside a glass wall, allowing visibility. The user exits the vehicle, and an operator guides them into the scanner. The parking area for the vehicle may not be air-conditioned but is separated by a gate that opens to allow the user to drive in and closes afterwards. Once the scan, which typically takes about 2-3 minutes, is completed, the user is instructed to get off the table, walk to their vehicle, and wait for the green light. The gate then opens, allowing the user to leave, and the next vehicle behind them drives in. To reduce scanner vacancy time the entrance can be accessed from both sides of the CT scan room to allow for entry while a patient is leaving to their car the other can be guided to the CT machine.

Additionally, the previously disclosed invention provides a method that includes providing a walk-through imaging area at the facility for users without vehicles. Walk-through users entering the facility on foot are guided to the imaging scanner via a dedicated entrance and pathway using directional signage and audio instructions. After performing the imaging scan on the walk-through user, they are directed to exit the imaging area through a separate exit pathway and doorway. The next walk-through user is allowed to enter the imaging area only after the previous walk-through user has exited, ensuring a smooth flow of foot traffic.

By streamlining the entire process, from patient recruitment, scheduling and preparation to the actual scanning procedure, the present invention significantly improves the overall patient experience and operational efficiency of imaging facilities compared to traditional methods.

The present invention is an improvement that provides a hybrid screening system that extends the automated walk-through medical imaging systems disclosed in the parent application into airport and controlled-access environments.

In one embodiment, the walk-through CT medical imaging system described in U.S. application Ser. No. 18/771,542 is incorporated herein and forms the basis of the medical imaging subsystem of the present invention. Such systems include automated subject positioning, rapid scan acquisition protocols, minimal or no onsite clinical staff, and AI-based image analysis for preventive screening. The present invention does not modify the underlying CT imaging technology disclosed in the parent application, but rather extends its deployment context and workflow integration.

In one embodiment, the system is deployed within an airport security environment and comprises a millimeter-wave security screening system configured to perform mandatory security scans on all passengers, a walk-through CT medical imaging system configured to perform voluntary low-dose medical imaging, a biometric identity verification system configured to authenticate passenger identity and opt-in consent, and a workflow orchestration and pathway control system configured to physically route passengers based on opt-in status. Security screening is always performed prior to any medical imaging.

In one embodiment, all passengers undergo mandatory security screening using existing millimeter-wave or equivalent non-ionizing technologies. These passengers entering the security zone are processed as follows: Security-Only Pathway Passengers who do not opt into medical imaging; Undergo standard security screening; Proceed directly to the sterile boarding area.

In one embodiment, passengers who opt into medical imaging undergo mandatory security screening, are routed via physical gates or signage to the walk-through CT imaging area, complete automated chest CT imaging, and rejoin the sterile boarding area without delaying standard security operations. The opt-in workflow may be managed through a mobile application, pre-registration system, or on-site biometric verification.

In one embodiment, the system employs biometric identity verification, including but not limited to ocular, facial, or fingerprint recognition, to authenticate passenger identity, verify informed consent for medical imaging, and associate medical imaging results with the correct individual. Consent is explicitly required and is separate from any security authorization.

In one embodiment, the system enforces strict separation between security data and medical data that security scan outputs are processed and disposed of according to security regulations and are not retained as medical data, medical CT images are processed, stored, and analyzed under medical data governance standards, and no security personnel access medical imaging data, and no medical personnel access security screening outputs.

In one embodiment, the walk-through CT subsystem performs low-dose imaging optimized for preventive screening, including imaging of the chest for cardiopulmonary assessment. Medical images are analyzed by an automated AI-based first-reader system, consistent with the disclosures of the parent application. Results may be delivered asynchronously to the participant through a secure digital platform. The medical imaging subsystem is never used for security threat detection, and the security screening subsystem is never used for medical analysis.

In certain embodiments, fusion of automatically measured height and weight with CT-derived anatomical measurements enables normalization of cardiopulmonary and metabolic metrics, thereby improving risk stratification relative to imaging alone.

In one embodiment, while described herein primarily in the context of airports, the system may be deployed in other controlled-access environments including, but not limited to: border crossings; maritime ports; railway terminals; stadiums and large venues; and government or corporate campuses.

In one embodiment, eligibility for voluntary medical imaging is determined prior to arrival at the controlled-access facility using a remote digital platform associated with the walk-through medical imaging system. A user may access a LIFEMAP application executing on a mobile device or computing platform, through which the user completes a pre-screening qualification process. The qualification process may include, without limitation, health questionnaires, age or risk-factor eligibility checks, prior imaging history, regulatory or protocol-based inclusion criteria, and determination of eligibility for initial or repeat medical imaging.

In another embodiment, biometric identity verification is used to authenticate passengers and confirm informed consent for voluntary medical imaging.

In one embodiment, upon completion of the qualification process, the system records eligibility status and associates it with the user's biometric identity. Once eligibility is confirmed, the LIFEMAP application generates a machine-readable digital token, such as a QR code, bar code, or equivalent, representing authorization for voluntary medical imaging. The digital token is cryptographically associated with the user's identity, encodes imaging eligibility and consent status, may include expiration timing or usage limits, and may include repeat-scan authorization parameters.

In one embodiment, at the controlled-access facility, the digital token is scanned upon arrival and prior to biometric identity verification, thereby enabling rapid association of the user with their pre-qualified screening status. The scanned token does not itself grant access to medical imaging, but serves as an initial workflow trigger. Final authorization for imaging is granted only after successful biometric identity verification, thereby preventing misuse, identity substitution, or unauthorized imaging.

Still in one embodiment, this pre-qualification and token-based workflow reduces on-site processing time, preserves security throughput, and ensures that only eligible individuals are routed to the hybrid screening pathway. A token scanning subsystem is co-located with the biometric identity verification system.

Security screening and medical imaging remain physically, functionally, and regulatorily separate, while being coordinated through a common workflow orchestration system.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### Embedded Multi-Spectrum Physiologic Imaging Subsystem

In certain embodiments, the walk-through CT medical imaging system further comprises an embedded physiologic imaging subsystem incorporating one or more visible-light, near-infrared (NIR), and infrared (IR) cameras configured to acquire three-dimensional anatomical and physiological data from the subject during passage through the scanning corridor.

The subsystem may include A structured-light or stereo visible-light 3D camera array, generating a depth-resolved surface model of the subject's body. Near-infrared cameras configured to extract blood-volume pulse signals based on photoplethysmographic (PPG) principles, enabling non-contact estimation of heart rate, heart-rate variability, respiration rate, blood oxygenation, and Infrared thermal imaging sensors configured to measure core-temperature-correlated facial thermal patterns peripheral perfusion distribution inflammatory dermal signatures.

In one embodiment, the multi-spectrum subsystem focuses on the face and upper thorax as the subject passes through the imaging area, using real-time landmark tracking and AI-based feature extraction to derive physiological parameters synchronized with CT acquisition.

### ECG Hand-Bar Subsystem for Arrhythmia Detection and ECG-Gating

In certain embodiments, the walk-through CT imaging corridor includes an ECG hand-bar module positioned overhead or laterally such that the subject briefly raises their hands and contacts insulated electrodes.

The hand-bar module provides Single-lead or dual-lead ECG acquisition and Real-time arrhythmia detection, including: tachycardia, bradycardia, atrial fibrillation,

Prospective or retrospective ECG-gating signals enabling cardiac CT imaging with compensation. Data from the ECG subsystem may be synchronized with the walk-through CT gantry rotation timing, physiologic signals from the visible/NIR/IR imaging subsystem, and height/weight measurement subsystems.

Integration With Opt-In Workflow and Mobile Application In certain embodiments, individuals who opt in via the LIFEMAP mobile application complete a pre-screening form that determines eligibility for physiologic signal acquisition, suitability for cardiac CT with ECG gating, whether repeat scans are medically indicated. Upon qualification, the LIFEMAP app generates a QR-code or digital token that is scanned at the airport prior to biometric verification. The token initiates a workflow sequence permitting biometric identity verification; routing to the CT area; activation of the embedded multi-spectrum cameras; activation of the ECG hand-bar module.

### Fusion of Physiologic and CT Data

In some embodiments, the system generates fused multi-modal outputs comprising CT-derived anatomical data, physiologic measurements (HR, HRV, SpO₂ trends, thermal maps), arrhythmia classification, 3D surface models, and ECG-gated cardiac imaging. These data may feed into an AI-based cardiometabolic risk engine to produce enhanced risk stratification.

### BRIEF DESCRIPTION OF DRAWINGS

The various exemplary embodiments of the present invention. which will become more apparent as the description proceeds, are described in the following detailed description in conjunction with the accompanying drawings, in which:

FIG. 1 illustrates a system overview diagram of a drive-through medical imaging system.

FIG. 2 depicts a layout of the imaging area.

FIG. 3 details the user flow with the associated mobile application and the mobile application's role in facilitating the CT scan.

FIG. 4 depicts an embodiment of the CT scan booth wherein the CT scan booth is designed to enable standing scans for the user.

FIG. 5 illustrates an embodiment of the drive-through medical imaging system that reduces scanner vacancy time.

FIG. 6 shows a hybrid system architecture integrating security screening and walk-through CT imaging.

FIG. 7 depicts passenger workflow illustrating security-only and hybrid opt-in pathways

FIG. 8 depicts physical layout of an airport security zone with integrated medical imaging lane.

FIG. 9 shows data governance and regulatory separation between security and medical systems.

FIG. 10 illustrates a walk-through CT imaging subsystem for voluntary chest imaging.

FIG. 11 illustrates physical routing elements.

FIG. 12 illustrates a biometric identity subsystem.

FIG. 13 illustrates an automated height-measurement subsystem integrated within the walk-through CT imaging pathway.

FIG. 14 illustrates a weight-measurement subsystem.

FIG. 15 is a data-flow diagram.

FIG. 16 is a flowchart illustrating a method for hybrid screening.

FIG. 17 illustrates a multi-spectrum physiologic imaging subsystem

FIG. 18 illustrates a physiologic signal extraction workflow.

FIG. 19 shows ECG Hand-Bar subsystem.

FIG. 20 illustrates integration of physiologic imaging+ECG with walk-through CT

FIG. 21 shows opt-in workflow with token+biometric+physiologic activation flow chart.

FIG. 22 illustrates an automated walk-through imaging corridor integrating CT acquisition with multi-spectrum physiologic sensing.

FIG. 23 depicts deployment of the hybrid security-medical imaging system within a controlled-access facility.

FIG. 24 illustrates a CT gantry with embedded multi-spectrum cameras and subject-centering mechanisms.

FIG. 25 shows a comprehensive system architecture integrating security workflow, CT imaging, physiologic subsystems, and AI analysis.

### DETAILED DESCRIPTIONS

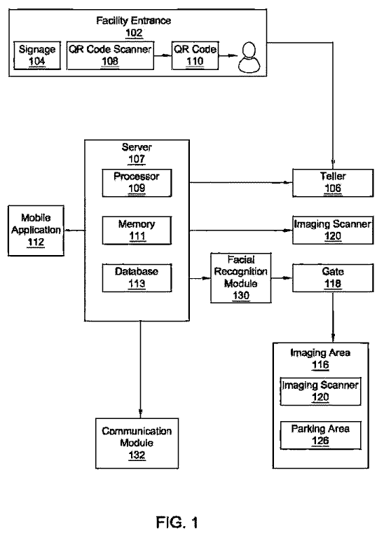

FIG. 1 illustrates a system overview diagram of a drive-through medical imaging system. The system comprises a facility entrance (102) equipped with signage (104) configured to guide a user in a vehicle, and a teller (106) for communicating with the user. In one embodiment, the teller (106) may be a human operator. In another embodiment, the teller (106) may be an automated artificial intelligence (AI) system that employs speech recognition and natural language processing techniques to interact with the user. The system also includes a server (107) equipped with a processor (109), memory (111), and a database (113) for storing and processing data related to the drive-through medical imaging system.

The automated AI system implemented as the teller (106) utilizes advanced speech recognition algorithms, such as deep learning-based models like convolutional neural networks (CNNs) and recurrent neural networks (RNNs). These models are trained on large datasets of speech samples, including various accents, dialects, and noise conditions, to achieve high accuracy and robustness. For example, a CNN architecture with multiple convolutional and pooling layers, followed by fully connected layers, can be employed for feature extraction and classification. The trained models are stored in the memory (111) and executed by the processor (109) of the server (107) to enable real-time speech recognition and processing.

The AI system also employs natural language processing (NLP) techniques, such as named entity recognition (NER), part-of-speech (POS) tagging, and dependency parsing, to analyze and understand the meaning and intent behind the user's spoken words. These techniques enable the system to extract relevant information and provide accurate responses. The NLP module is built using state-of-the-art deep learning architectures, such as the Transformer model, which has been pre-trained on large text corpora and fine-tuned for the specific domain of medical imaging inquiries. The NLP models and associated data are stored in the database (113) and accessed by the processor (109) for real-time natural language understanding and generation. The system further includes a QR code scanner (108) disposed at the entrance (102), adapted to read a QR code (110) provided to the user.

Additionally, the system comprises a mobile application (112), configured to allow the user to answer questions, receive a physician order, schedule an imaging scan appointment, and access instructions for preparing for the scan. The mobile application (112) is developed using modern healthcare informatics software development frameworks, such as FHIR (Fast Healthcare Interoperability Resources), and is available for both iOS and Android operating systems. It integrates with the overall drive-through medical imaging system to enhance user experience and streamline the process, serving as a central hub for users to input necessary information, receive personalized guidance, and manage their appointments. The application (112) exchanges data with the system's backend server (107) using secure communication protocols, such as HTTPS and SSL/TLS, and robust encryption mechanisms to protect sensitive patient information. The server (107) stores and manages the data exchanged with the mobile application (112) in its database (113).

Upon the QR code (110) being read by the scanner (108), a green sign (114) is activated to guide the user to proceed to an imaging area (116) located inside the facility. The imaging area (116) is separated from the entrance (102) by agate (118). The gate (118) is configured to open to allow the user's vehicle to enter the imaging area (116) and to close during the imaging scan. The server (107) controls the operation of the gate (118) based on the QR code (110) information and the status of the imaging process. The imaging equipment can be standing or a standard flat table CT scanner.

The imaging area (116) includes an imaging scanner (120), such as a CT scanner, that is visible to the user through a glass wall (124). The CT scanner may employ advanced X-ray imaging technology, such as multi-slice CT or dual-energy CT, to produce high-resolution, detailed images of the patient's anatomy. The scanner (120) may also incorporate dose reduction techniques, such as iterative reconstruction algorithms or adaptive dose modulation, to minimize radiation exposure to the patient. The scanner (120) is connected to the server (107), which controls its operation and receives the acquired imaging data for further processing and storage in the database (113).

The area (116) also comprises a parking area (126) adapted to accommodate the user's vehicle and an operator (128) who is configured to guide the user from their vehicle to the scanner (120). The parking area (126) may include sensors, such as ultrasonic sensors or cameras, to assist the user in properly positioning their vehicle and ensuring a safe and efficient parking process. The sensor data is transmitted to the server (107) for analysis and guidance generation. After the imaging scan is completed within a pre-determined duration, the user is signaled to return to their vehicle, and the gate (118) opens upon a green light signal controlled by the server (107) to allow the vehicle to exit. The system is configured to allow entry of another user's vehicle after the previous user's vehicle has exited, ensuring a smooth and continuous flow of patients through the drive-through imaging process.

The system includes additional key components, such as a facial recognition module (130) for verifying the user's identity before allowing vehicle entry. The facial recognition module (130) may utilize advanced computer vision algorithms and deep learning techniques to accurately match the user's face with their pre-registered facial data, providing an additional layer of security and ensuring that only authorized individuals can access the imaging services. The facial recognition models and associated data are stored in the database (113) and executed by the processor (109) for real-time identity verification.

Furthermore, the system includes a communication module (132) for electronically transmitting the imaging scan results to a physician upon completion. The communication module (132) may utilize secure healthcare data exchange standards, such as HL7 or DICOM, to ensure the integrity and confidentiality of the transmitted imaging data. The module (132) may also incorporate encryption and authentication mechanisms to prevent unauthorized access to the sensitive medical information. The server (107) manages the operation of the communication module (132) and facilitates the secure transmission of imaging data from the database (113) to the designated physician.

In one embodiment, the signage (104) at the facility entrance (102) may comprise an interactive display adapted to answer user questions. The interactive display may utilize touchscreen technology and natural language processing to provide users with relevant information, such as directions, wait times, or frequently asked questions about the imaging process. This feature enhances the user experience and reduces the workload on human operators. The interactive display is connected to the server (107), which processes the user queries and generates appropriate responses using the NLP models and data stored in the database (113).

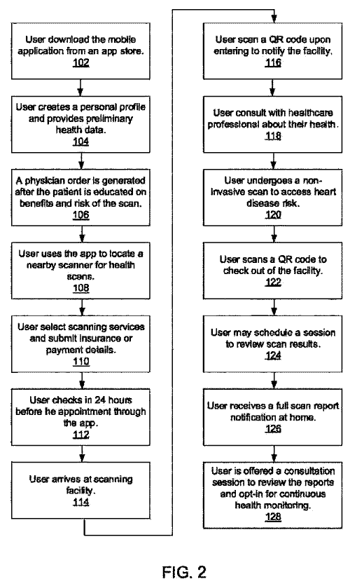

FIG. 2 details the user flow with the associated mobile application (112) and the mobile application's (112) role in facilitating the CT scan, according to an embodiment of the present invention. The method begins with the user downloading the mobile application (“app”) (112) from an app store at step 102. In one embodiment, the user may access the app's (112) download page directly by scanning a quick response (QR) code (110) displayed on a television, monitor, or other QR code display.

Upon launching the app (112) for the first time, the user creates a personal profile at step 104. During the profile creation process, the app (112) collects preliminary health data by presenting the user with a series of questions related to cardiovascular and lung cancer risk factors. The user's responses to these questions enable the app (112) to tailor the subsequent scanning process to the user's specific health needs. In one embodiment, the app (112) may utilize a natural language processing (NLP) engine, such as Google's Dialogflow, to interpret the user's responses and extract relevant health information.

At step 106, the user employs the app's (112) built-in scanner locator feature to find a nearby affiliated scanner (120) capable of performing the desired health scans. Once a suitable scanner (120) has been identified, the user may proceed to schedule an appointment through the app's (112) integrated scheduling interface.

The user selects the specific scanning services required and provides their insurance details at step 108. In the event that the user does not have insurance coverage, the app (112) presents an alternate path at step 110, prompting the user to enter their credit card information to pay for the selected services. The app (112) may employ a secure payment gateway, such as Stripe or PayPal, to process the payment transaction.

At step 112, the user is required to check-in via the app (112) 24 hours prior to their scheduled appointment. This check-in process serves to confirm the user's appointment and allows the scanning facility to prepare for the user's visit, thereby improving the overall efficiency of the service.

On the day of the scheduled scan, the user arrives at the designated facility at step 114. Upon entering the facility, the user scans a QR code (110) at step 116, which automatically checks the user in and notifies the facility's staff to prepare for the user's scheduled service. The app (112) may leverage the device's built-in camera and a QR code scanning library, such as AV Foundation, to facilitate this process. At step 118, the user engages in a consultation with a healthcare professional. During this consultation, the user may discuss their health concerns, review their medical history, and receive any necessary preparations for the upcoming scan based on the information provided in their app (112) profile. The healthcare professional may access the user's profile information through a secure, HIPAA-compliant web portal that integrates with the app's (112) backend infrastructure.

The user undergoes the Automatic Coronary Artery Calcium (Auto-CAC) scanning process at step 120. This non-invasive scan, which measures the calcium content in the user's arteries as an indicator of heart disease, is designed to be quick and comfortable. The Auto-CAC scan requires no needles, contrast injections, or hospital gowns, and is capable of completing 10 different tests in under 30 seconds. The scan results are automatically uploaded to the user's profile in the app's (112) secure, cloud-based database.

In certain embodiments, the Auto-CAC scan is performed autonomously without necessitating human intervention. User input pertaining to the scan type and any associated parameters is acquired from the patient or the associated healthcare professional via the mobile application (112) executing on the personal computing device, which may comprise a mobile phone or laptop. The user input is securely transmitted from the personal device to the communication module (132) employing healthcare data exchange standards, such as Health Level Seven (HL7) or Digital Imaging and Communications in Medicine (DICOM), to ensure the integrity and confidentiality of the transmitted data. The imaging scanner (120) subsequently utilizes the transmitted data to automatically execute the selected scans based on the received parameters, alleviating the need for onsite programming.

Upon completion of the Auto-CAC scan, the user scans a QR code (110) to check out of the facility at step 122. This check-out process logs the completion of the appointment and may trigger associated billing processes or final administrative tasks.

At step 124, the user has the option to schedule a complimentary review session with a health coach. During this review session, the health coach assists the user in interpreting their scan results and discusses the implications of these results in the context of the user's overall health. The health coach may also provide guidance on how to effectively communicate the scan results to the user's primary care physician.

The user returns home at step 126. Once at home, the user receives a notification from the app (112) indicating that their full scan report and any additional notes or recommendations from the healthcare professional are available for viewing within the app (112). The app (112) may utilize push notification services, such as Apple Push Notification Service (APNs) or Firebase Cloud Messaging (FCM), to deliver these notifications to the user's device.

Finally, at step 128, the user is presented with the option to become a member of an associated community facilitated by the mobile app (112). Membership in this community grants the user access to personalized health plans and ongoing support aimed at preventing serious health issues, such as heart attacks and lung cancer. The app (112) may integrate with a customer relationship management (CRM) platform, such as Salesforce or HubSpot, to manage the user's membership and facilitate ongoing communication and support.

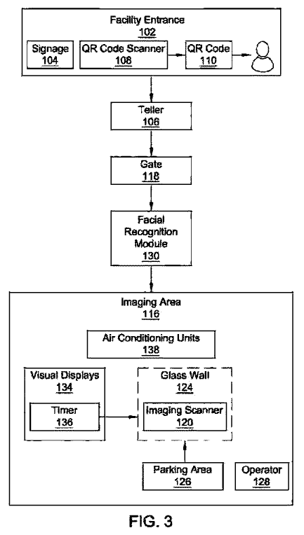

FIG. 3 illustrates an aerial view of the floor plan for a drive-through medical imaging system. The floor plan depicts a facility entrance (102) equipped with signage (104) configured to guide a user in a vehicle, and a teller (106) for communicating with the user. The teller (106) may be a human operator or an automated AI system that employs speech recognition and natural language processing techniques to interact with the user. The system further includes a QR code scanner (108) disposed at the entrance (102), adapted to read a QR code (110) provided to the user.

The floor plan also shows an imaging area (116) located inside the facility, separated from the entrance (102) by a gate (118). The gate (118) is configured to open to allow the user's vehicle to enter the imaging area (116) and to close during the imaging scan. The imaging area (116) includes an imaging scanner (120), such as a CT scanner (122), that is visible to the user through a glass wall (124). Within the imaging area (116), a parking area (126) is provided, adapted to accommodate the user's vehicle. An operator (128) is stationed near the scanner (120) to guide the user from their vehicle to the scanner (120) and back. Visual displays (134) are mounted in clear view, showing a scan duration timer (136) that counts down the time remaining, typically 2-3 minutes. The displays (134) also provide green light signals to indicate when the user should exit the scanner (120) and return to their vehicle.

Arrows on the ground indicate the intended flow and path of the user from the parking area (126) to the scanner (120) and back to their vehicle after the scan is complete. The system is configured to allow entry of another user's vehicle after the previous user's vehicle has exited, ensuring a smooth and continuous flow of patients through the drive-through imaging process.

The floor plan also includes additional key components, such as a facial recognition module (130) for verifying the user's identity before allowing vehicle entry, and air conditioning units (138) for user comfort.

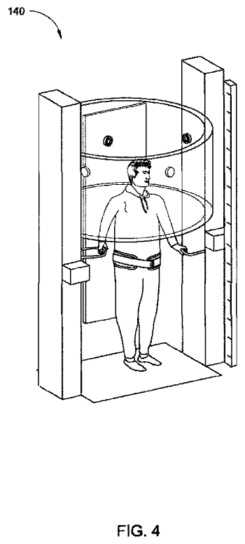

FIG. 4 depicts an embodiment of the CT scan booth (140) wherein the CT scan booth (140) is designed to enable full-body weight-bearing CT scans for the user in a standing position. The booth (140) is an enclosure that allows a user to exit their vehicle and enter for a scan. An external view of the booth (140) shows its compact footprint of approximately 1×1 meter and clearly marked entrance.

The interior of the booth (140) is equipped with sturdy handles (142), positioned for the user to grasp onto to maintain a steady posture during the scan. A visual light signal and audio guide (144) are used to instruct the user on proper positioning and when to exit the booth (140) upon completion of the scan. The system's software enables automatic reconstruction of the 3D volumetric data into multiplanar images for visualization and analysis. The CT scanner includes multiple cameras to view the subject's position accurately and to enable the AI to manage the CT scanning by interacting with the subjects and guiding them to properly position for the CT scan. Also to answer any questions they might have. The AI just like a human radiology tech will be able to answer questions necessary for conducting the CT scan. Additionally the CT scan platform that has an elevator component on the base to elevate the standing subject's chest to center of the CT imaging field of view includes tools to measure the subject's weight and height. The weight is measured using scale electronics inside the base of the elevator platform, and the height is measures using the cameras inside the CT scan and the distance from the step-on elevator platform.

The standing weight-bearing CT scan booth (140) may be deployed at various locations where the drive-through or walk-through medical imaging system is implemented, such as hospitals, clinics, and dedicated imaging facilities. The booth's (140) modular design allows it to be easily installed as an add-on to existing CT or other imaging systems, expanding the range of services offered to include weight-bearing scans for musculoskeletal and other applications. The compact footprint and lower radiation dose compared to conventional CT make it suitable for use in a variety of settings.

In another embodiment of the drive through medical imaging system, a walk-through embodiment is implemented which parallels the drive through system but in a walk-through experience. The system comprises a facility entrance (102) equipped with signage (104) configured to guide a user on foot. An automated AI system employs speech recognition and natural language processing techniques to interact with the user, utilizing advanced algorithms such as CNNs, RNNs, and NLP techniques like named entity recognition NER, POS tagging, and dependency parsing.

The system further includes a QR code scanner (108) disposed at the entrance (102), adapted to read a QR code (110) provided to the user via a mobile application (112). The mobile application (112), developed using modern healthcare informatics software development frameworks like FHIR (Fast Healthcare Interoperability Resources), allows the user to answer questions, receive a physician order, schedule an imaging scan appointment, and access instructions for preparing for the scan.

Upon the QR code (110) being read by the scanner (108), a green sign (114) is activated to guide the user to proceed to a walk-through imaging area (146) located inside the facility. The walk-through imaging area (146) is separated from the entrance (102) by an automated gate (118), configured to open to allow the user to enter the walk-through imaging area (146) and to close during the imaging scan.

The walk-through imaging area (146) includes an advanced imaging scanner (120), such as a multi-slice CT or dual-energy CT scanner, that is visible to the user through a glass wall (124). The scanner (120) employs dose reduction techniques, such as iterative reconstruction algorithms or adaptive dose modulation, to minimize radiation exposure to the patient.

The walk-through imaging area (146) also comprises a waiting area adapted to accommodate the user and an automated guidance system (148) to guide the user from the waiting area to the scanner (120). After the imaging scan is completed within a pre-determined duration, the user is signaled to return to the waiting area, and the gate (118) opens upon a green light signal to allow the user to exit.

The system includes a facial recognition module (130) for verifying the user's identity before allowing entry, utilizing advanced computer vision algorithms and deep learning techniques. A communication module (132) electronically transmits the imaging scan results to a physician upon completion, employing secure healthcare data exchange standards like HL7 or DICOM, along with encryption and authentication mechanisms.

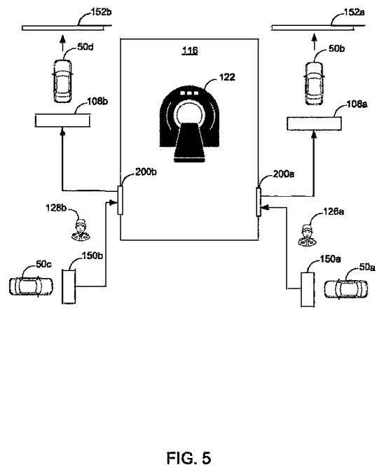

FIG. 5 illustrates an additional embodiment of the drive-through medical imaging system that reduces scanner vacancy time. In this embodiment, the imaging area (116) is accessible from both sides, with separate entrance (150) and exit (152) gates. This configuration allows a first user's vehicle to enter the imaging area (116) via the entrance gate (150) while a second user's vehicle that has completed the imaging scan exits through the exit gate (152). The entrance gate (150) and exit gate (152) operate independently, allowing the next user to be guided by the operator (128) to the CT scanner (122) while the previous user returns to their vehicle and exits the imaging area (116). This simultaneous entry and exit of users' vehicles through the separate gates (150, 152) reduces scanner vacancy time and improves the efficiency of the drive-through medical imaging process. The system's automated control module coordinates the opening and closing of the gates (150, 152) and the guidance provided by the operator (128) to ensure a seamless and continuous flow of users through the imaging area (116).

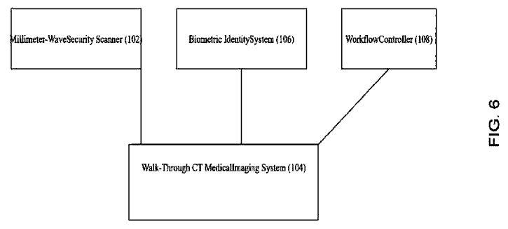

FIG. 6 is a block diagram illustrating a hybrid screening system deployed within a controlled-access environment. The system includes a millimeter-wave security screening system (102), a walk-through computed tomography (CT) medical imaging system (104), a biometric identity verification system (106), a workflow control system (108), and physical pathway routing elements (110). The workflow control system (108) enforces mandatory sequencing such that all subjects undergo millimeter-wave security screening prior to access to the CT medical imaging system.

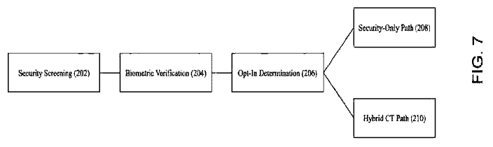

FIG. 7 is a flow diagram illustrating passenger progression through the hybrid screening system. The diagram illustrates an initial security screening stage (202), a biometric identity verification stage (204), an opt-in determination step (206), a divergence into either: a security-only pathway (208), or a hybrid security+medical imaging pathway (210). Passengers routed to the hybrid pathway undergo walk-through CT imaging after successful completion of security screening.

Still in FIG. 7, opt-in determination may be performed in advance via a mobile application, and that a digital token generated by the application is scanned upon arrival at the screening environment to initiate the hybrid pathway workflow.

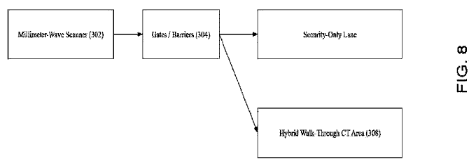

FIG. 8 is a top-down schematic view of an airport checkpoint illustrating physical deployment of millimeter-wave scanners (302), controlled pathway gates or barriers (304), signage and directional indicators (306), and a walk-through CT imaging area (308). The figure illustrates that the hybrid medical imaging pathway operates without impeding throughput of the security-only pathway.

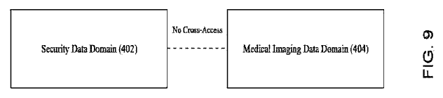

FIG. 9 is a logical diagram illustrating separation between security data processing and storage (402) and medical imaging data processing and storage (404). The workflow control system (406) enforces access controls preventing security personnel from accessing medical imaging data and preventing medical systems from accessing security scan outputs.

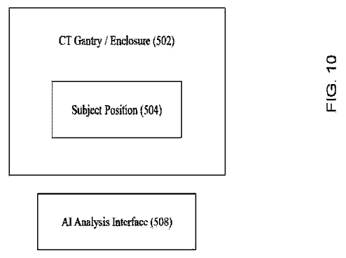

FIG. 10 shows a gantry or enclosure suitable for standing subjects (502), automated subject positioning components (504), a CT acquisition subsystem (506), and an interface with an AI-based image analysis engine (508). No security threat detection components are included within the CT subsystem.

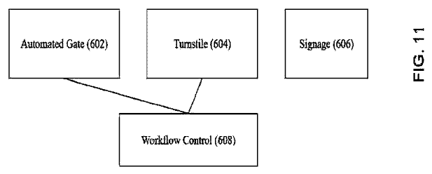

FIG. 11 illustrates physical routing elements including automated gates (602), turnstiles or barriers (604), directional signage (606), and their coordination under control of the workflow system (608) to direct subjects into appropriate screening paths.

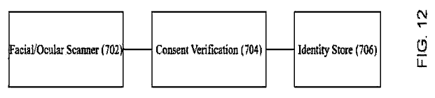

FIG. 12 illustrates a biometric identity subsystem comprising biometric capture devices (702) including facial or ocular scanners, a consent verification module (704), a secure identity datastore (706). The subsystem enables medical imaging only upon confirmation of voluntary opt-in consent.

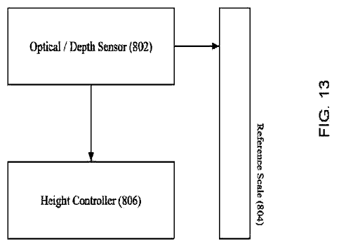

FIG. 13 illustrates an automated height-measurement subsystem integrated within the walk-through CT imaging pathway. The subsystem includes optical or depth-sensing elements (802), a reference frame or vertical scale (804), a controller (806) that determines subject height during passage through the system. No additional subject interaction beyond normal walking or standing is required.

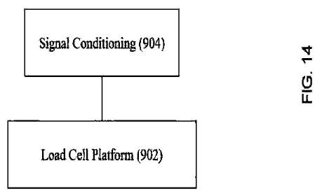

FIG. 14 illustrates a weight-measurement subsystem comprising: load cells or pressure sensors integrated into a platform or floor (902); a signal conditioning module (904); and synchronization with CT acquisition timing (906). The subsystem acquires weight measurements while the subject is positioned for medical imaging.

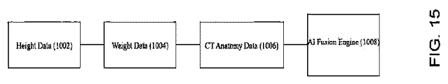

FIG. 15 is a data-flow diagram illustrating integration of height data (1002), weight data (1004), CT-derived anatomical measurements (1006), within an AI-based analysis engine (1008) configured to compute derived metrics such as body mass index (BMI) and cardiometabolic risk indicators.

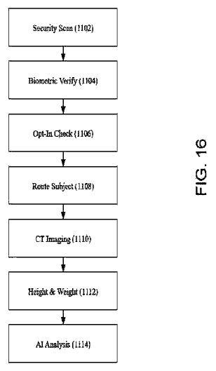

FIG. 16 is a flowchart illustrating a method for hybrid screening including performing millimeter-wave security screening (1102), verifying biometric identity (1104), determining opt-in status (1106), routing the subject (1108), performing walk-through CT imaging (1110), acquiring height and weight measurements (1112), and analyzing medical imaging data using AI (1114).

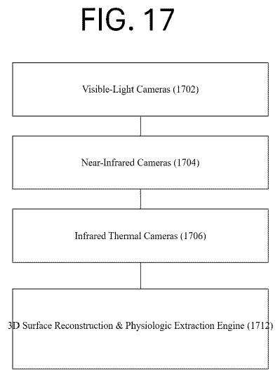

FIG. 17 shows a perspective diagram illustrating visible-light cameras (1702), NIR cameras (1704), IR thermal imagers (1706), facial tracking region (1708), structured-light/depth projector (1710), and 3D surface reconstruction module (1712).

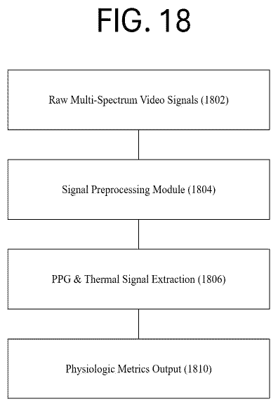

FIG. 18 is a physiologic signal extraction workflow that includes a block diagram showing acquisition of raw visible/NIR/IR signals (1802), preprocessing module (1804), PPG-based physiological signal extraction (1806), thermal analytics engine (1808), and physiologic parameter output (1810).

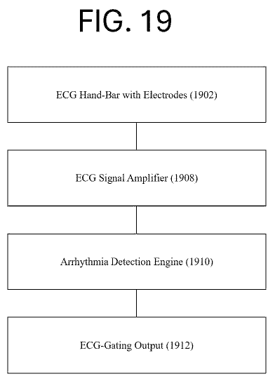

FIG. 19 a diagram showing overhead or lateral-mounted ECG hand-bar (1902), electrode contact points (1904), subject hand placement (1906), ECG amplifier module (1908), arrhythmia detection engine (1910), and ECG-gating output (1912).

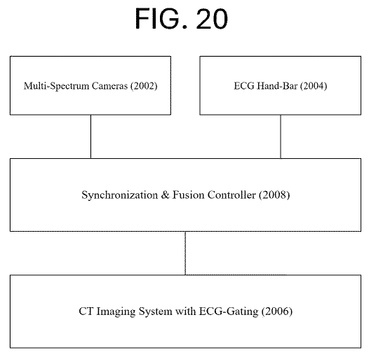

FIG. 20 shows a system diagram illustrating synchronized timing between multi-spectrum cameras (2002), ECG hand-bar (2004), CT gantry rotation (2006), and AI fusion engine (2008) producing multi-modal outputs (2010).

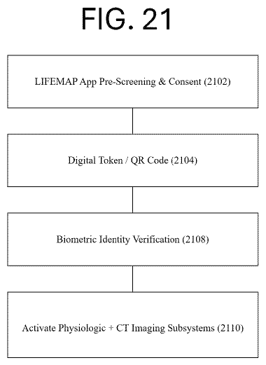

FIG. 21 is a flowchart showing user qualification in the LIFEMAP app (2102), digital token generation (2104), token scan at airport (2106), biometric ID verification (2108), activation of physiologic subsystems (2110), and routing to CT imaging (2112).

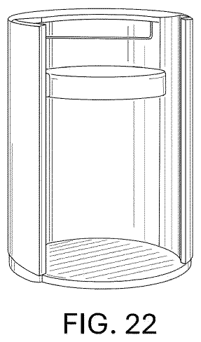

FIG. 22 shows Automated Walk-Through Imaging Corridor With Integrated Multi-Spectrum Sensing illustrates an embodiment of the automated walk-through imaging corridor incorporating the CT gantry, embedded visible-light/NIR/IR camera arrays, overhead ECG hand-bar, and synchronized subject-tracking system. The corridor includes directional pathway indicators guiding the subject through sequential biometric verification, multi-spectrum physiologic acquisition, and CT imaging. Cameras mounted along the corridor capture 3D surface geometry and physiologic signals, while the ECG hand-bar provides cardiac rhythm and gating signals. The system operates autonomously without real-time human intervention, coordinating positioning, timing, and physiologic synchronization for high-throughput preventive screening.

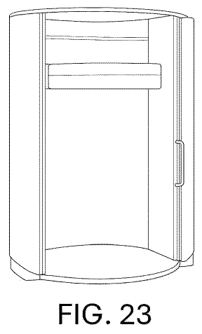

FIG. 23 shows Hybrid Security-Medical Screening Deployment Within Controlled-Access Environment. FIG. 23 depicts an integrated deployment of the hybrid screening system within an airport or similar controlled-access facility. The layout includes the millimeter-wave security scanners, controlled routing barriers, token-scanning stations, biometric verification points, and the optional CT medical imaging lane. The diagram shows separation between the mandatory security-only pathway and the voluntary medical imaging pathway, as well as the workflow control architecture that preserves security throughput while allowing opt-in CT screening for eligible subjects. Physical gates and signage are coordinated by the workflow system to maintain regulatory separation between security operations and medical imaging data governance.

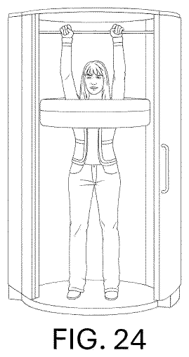

FIG. 24 shows CT Gantry With Embedded Physiologic Imaging and Subject-Centering Mechanisms. FIG. 24 illustrates a detailed view of the CT gantry configured for walk-through imaging, showing embedded multi-spectrum cameras, structured-light projectors, internal subject-centering markers, and optional platform-integrated height/weight measurement sensors. The embedded cameras simultaneously acquire visible-light, NIR, and IR data for physiologic signal extraction, while CT acquisition components generate low-dose anatomical images optimized for cardiopulmonary screening. The figure highlights geometric alignment features and synchronization modules enabling unified reconstruction of CT images with physiologic parameters.

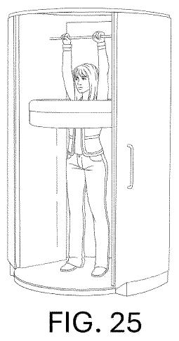

FIG. 25 shows a Comprehensive System Architecture Integrating CT Imaging, Physiologic Modules, Security Workflow, and AI Analysis Engine. FIG. 25 presents a high-level system architecture diagram showing the interaction between subsystems: the millimeter-wave security screening subsystem, biometric identity subsystem, digital token verification subsystem, walk-through CT imaging unit, physiologic imaging subsystem (visible/NIR/IR cameras and ECG hand-bar), height/weight measurement modules, workflow control system, and the AI-based first-reader analysis engine. Data flows depict how security data and medical data remain segregated through independent pipelines, while the workflow controller orchestrates subject routing, timing, and subsystem activation to produce AI-derived preventive health assessments.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

We claim:

1. A screening system comprising:
a walk-through computed tomography (CT) medical imaging system configured to acquire medical imaging data from a human subject;
a camera system using visible light, near infrared and infrared imaging sensors to image the human body in 3D;
a non-ionizing millimeter-wave security screening system configured to screen the human subject for concealed security threats;
an identity verification system configured to authenticate an identity of the human subject and confirm qualifications and voluntary opt-in consent for medical imaging;
a workflow control system configured to:
require the human subject to undergo security screening using the millimeter-wave security system, and
selectively route the human subject to the walk-through CT medical imaging system only upon verification of qualification and voluntary opt-in for medical imaging; and physical pathway routing elements that guide the human subject into either a security-only pathway or a hybrid security-plus-medical imaging pathway.

2. The system of claim 1, wherein the workflow control system enforces a mandatory sequence in which security screening occurs prior to any medical CT imaging.

3. The system of claim 1, wherein data generated by the non-ionizing millimeter-wave security screening system is processed and governed under security regulations, and data generated by the walk-through CT medical imaging and visible light imaging system is processed and governed under medical data regulations.

4. The system of claim 1, wherein the workflow control system disables the walk-through CT medical imaging system unless explicit opt-in consent and qualification for medical imaging have been authenticated using the identity verification system.

5. The system of claim 1, wherein the identification, qualification and consent are performed in advance via a mobile application that generates a QR code for rapid authentication and seamless workflow.

6. The system of claim 1, further comprising a multi-spectrum physiologic imaging subsystem including visible-light, near-infrared, and infrared cameras configured to generate a three-dimensional surface model of the subject and extract physiological signals during data acquisition within the walk-through imaging system.

7. The system of claim 1, wherein the physiological signals extracted from the multi-spectrum imaging subsystem include heart rate, heart-rate variability, blood oxygenation, respiration rate, or temperature-derived biomarkers.

8. The system of claim 1, further comprising an ECG hand-bar module including one or more contact electrodes configured to acquire ECG signals when the subject raises their hands to the bar.

9. The system of claim 3, wherein ECG signals are used to detect arrhythmias selected from tachycardia, bradycardia, or atrial fibrillation and to provide ECG-gating inputs for cardiac CT image acquisition.

10. The system of claim 1, wherein the multi-spectrum imaging subsystem and the ECG hand-bar module operate synchronously with CT acquisition to generate fused anatomical and physiological datasets for AI-based cardiometabolic risk assessment.

11. The system of claim 1, further comprising selective routing of opt-in subjects, which does not reduce throughput of subjects undergoing security-only screening.

12. The system of claim 1, wherein medical imaging data acquired by the walk-through CT medical imaging system is analyzed using an automated AI-based first-reader system without real-time human intervention.

13. The system of claim 1, wherein the walk-through visible light, near infrared and infrared imaging system analyzes biological signals from skin and outer surface of human body.

14. The system of claim 8, wherein the biological signals can provide information on the subject's body temperature, heart rate, blood oxygenation, blood pressure, skin pathologies and other health related information.

15. The system of claim 1, wherein the walk-through CT medical imaging system operates at a radiation dose suitable for preventive health screening.

16. The system of claim 1, wherein the entire system generates hybrid outputs combining the output of each component of the system.

17. The system of claim 1, further comprising physical pathway routing elements selected from the group consisting of gates, barriers, or signage that guide the human subject into either a security-only pathway or a hybrid security-plus-medical imaging pathway.

18. The system of claim 1, wherein the system is deployed within an airport security checkpoint environment.

19. The system of claim 1, wherein the system is deployed within one or more controlled-access facilities selected from border crossings, seaports, railway stations, stadiums, corporate campuses, or government facilities.

20. The system of claim 1, further comprising a height-measurement subsystem configured to automatically determine height of the human subject during passage through the walk-through CT medical imaging system.

21. The system of claim 1, further comprising a weight-measurement subsystem configured to automatically determine weight of the human subject, wherein the human subject is positioned within the walk-through CT medical imaging system.

22. The system of claim 1, wherein height and weight measurements are obtained without requiring additional subject interaction beyond participation in the security screening and voluntary medical imaging workflow.

23. The system of claim 13, wherein the workflow control system or AI-based analysis system computes a body mass index (BMI) metric using the automatically determined height and weight.

24. The system of claim 20 and 21, wherein height and weight are measured using one or more of load cells integrated into a platform or floor, optical or depth sensors, stereoscopic imaging, structured light, lidar, or pressure sensors.

25. The system of claim 13, wherein height and weight measurements are temporally synchronized with CT image acquisition.

26. The system of claim 14, wherein height, weight, and CT-derived anatomical measurements are combined by the AI-based first-reader system to improve cardiometabolic or pulmonary risk assessment.

27. The system of claim 1, wherein eligibility for voluntary medical imaging is determined prior to arrival at the screening environment using a remote application executing on a user device.

28. The system of claim 18, wherein the remote application generates a machine-readable digital authorization token representing eligibility for voluntary medical imaging.

29. The system of claim 19, further comprising a token scanning subsystem configured to scan the digital authorization token at the screening facility prior to biometric identity verification.

30. The system of claim 20, wherein access to the walk-through CT medical imaging system is enabled only after biometric identity verification confirms correspondence between the scanned digital authorization token and the individual.

31. The system of claim 18, wherein the remote application determines eligibility for repeat medical imaging based on at least one of elapsed time since a prior scan or predefined screening eligibility criteria.

32. A method for hybrid screening of a human subject, comprising:
performing non-ionizing security screening of the human subject using a millimeter-wave security system;
verifying biometric identity of the human subject;
determining opt-in status of the human subject for medical imaging;
routing the human subject, upon verified opt-in, to a walk-through CT medical imaging system via physical routing elements;
acquiring medical imaging data using the walk-through CT medical imaging system;
analyzing the medical imaging data using an automated AI-based system for risk assessment.

33. The method of claim 32, further comprising automatically determining height and weight of the human subject during the walk-through CT medical imaging process.

34. The method of claim 32, wherein the method is performed within an airport security screening environment.
